(19) United States
(12) Patent Application Publication
(10) Pub. No.: US 2005/0020871 A1
(43) Pub. Date: Jan. 27, 2005

(54) Artificial contractile tissue

(76) Inventors: Piergiorgio Tozzi, Lausanne (CH); Ludwig Karl Von Segesser, Lausanne (CH)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/893,907

(22) Filed: Jul. 20, 2004

Related U.S. Application Data
(60) Provisional application No. 60/488,792, filed on Jul. 21, 2003.

(51) Int. Cl. A61M 1/12; A61F 2/08

(52) U.S. Cl. 600/17; 623/14.13; 623/3.11

(57) ABSTRACT

An artificial contractile tissue and a cardiac assist device and method, in which a spatial arrangement of contractile elements defining a three-dimensions contractile web is driven in a reversible contraction in order to determine a controlled contraction of the web in at least one direction.

### BACKGROUND OF THE INVENTION

The present invention relates to an artificial contractile tissue generally devised to be used in the medical field.

More specifically, the artificial contractile tissue reproduces the mechanical proprerties of human muscles and can be used to restore or improve the contractile performance of any biologic muscle such as the cardiac muscle or the skeletal muscle.

One of the applications of this invention consists of wrapping the whole heart or a part of it in such a way to increase its capacity to pump the blood into vessels. Therefore, the described invention further relates to heart assist devices.

In particular, the artificial contractile tissue can be easily used to assist the atrial contraction of patients with atrial fibrillation.

As it is well known, atrial fibrillation is the most common abnormal heart rhythm. Patients suffering from chronic atrial fibrillation account for more than 2 million people in the United States with an annual incidence of 400.000 new cases each year. Atrial fibrillation consists of a very fast, uncontrolled heart rhythm caused when the upper chambers of the heart (the atria) quiver, instead of beating. Because the pumping function of the upper cardiac chambers isn't working properly, the heart is less effective, minus 15% of its pumping capacity, and blood pools in the atria and clots. In about 5% of patients, clotted blood dislodges from the atria and results in a stroke. Therefore, patients with chronic atrial fibrillation suffer from cardiac failure and are at high risk of thrombo-embolic accidents. The treatment of atrial fibrillation consists primarily of palliation, mostly in the form of pharmacological intervention aiming to reduce the risk of stroke using anticoagulant agents. At present, the only treatment which potentially cures the atrial fibrillation is the “Maze” procedure, consisting in a surgical intervention compartimentalizing both atria and creating a labyrinth with blind alleys which help to restore the correct electric transmission and the correct functioning of the atrial contraction.

Unfortunately, the rate of restoring atrial contraction is quite low varying from 21 to 75% depending on the aetiology of the atrial fibrillation and patients require anticoagulation therapy for life anyhow, being exposed to the risk of hemorrhagic complications. Moreover, even if sinus rhythm is restored, atria have definitively lost their capability to coordinate their contraction in order to push the blood into the ventricles (lower chambers of the heart) producing an efficient transport function, thus the hemodynamic gain is negligible. Therefore, the atrial kick is compromised both in patients with persistent atrial fibrillation and in patients in which the sinus rhythm has been restored with the Maze procedure.

In addition, the Maze procedure has the drawback to involve an invasive intervention on the atria tissue and which may also involve the temporarely application of a post-intervention pace maker.

Another clinical situation in which the artificial contractile muscle can play a therapeutical role is the Fontan circulation. The Fontan circulation is a surgical direct connection between the systemic veins and the pulmonary artery in patients born with tricuspid valve atresia or one and a half ventricle. Consequently, the pulmonary flow depends only on the gradient between right and left atrial pressure. In patients with increased pulmonary resistance, the surgical procedure of atrio-pulmonary connection is bound to fail. Since there are no other therapeutical options, in these patients there is the need to increase the atrial pulmonary flow after the Fontan operation.

Heart compression is a physiologic way to mechanically support the heart function because there is no need for artificial valves and anticoagulation therapy. Currently available pneumatic systems have tubes piercing the skin increasing the risk of infections, heavy power supplies (˜700 watt) requiring patients to be constantly tethered to an external power source and need anticoagulation therapy.

In view of the above it is evident the need for an effective cardiac contraction assist, and in particular for an assist to restore atrial transport function in patients with atrial fibrillation, as well as an assist device to create an atrial kick for patients subjected to Fontan circulation.

Extension of this technology to the treatment of congestive heart failure could represent one more application of the artificial contractile tissue. Other applications in the medical field are the treatment of neuromuscular diseases causing paralysis and post traumatic paralysis of lower and/or upper extremities, to increase muscular strength, to treat sexual impotence, etc.

### SUMMARY OF THE INVENTION

The aim of the present invention is to solve the above identified problems in the prior art by providing both a reliable and fully implantable cardiac contraction assist device and a method based on a low-invasive approach based on a physiological support of the mechanical function of the heart without the need of arftificial valves, magnetic propeller and anticoagulation therapy.

The aim has been reached according to the present invention by a cardiac contraction assist device and method using an artificial beating tissue based on nanotechnology actuators as source of one or more spatially oriented forces which are used to exert an extra pressure on the cardiac region to be assisted.

The novel method and device of the invention is designed to organize contractile elements deformation into a three dimensional movement reproducing the beahviour of biological muscles.

Preferably, the individual contractile elements can be made of Shape Memory Alloy elements (SMA), Electro Active Polymer elements (EAP), Electroactive Ceramic elements (EAC), carbon nanotubes (CNT), piezoelectric materials or other suitable material.

More particularly, the invention may provide contractile units capable of undergoing electrically controllable large longitudinal deformations.

Several elements or units can be embedded in an elastomeric material in such a way to create a web. Activation of the contractile elements causes a reduction in their lenght that is associated to the contraction of the web.

The spatial organization of the contractile elements is used to develop optimized contractile force per weight and space used. To this end, serial and/or parallel connections have also been considered to achieve the necessary performance characteristics in terms of lenght of displacement and of power of contraction.

Advantageously, the device can be fashioned as a beating tissue having different dimensions and shapes (potentially without restrictions) in order to accomplish different needs.

In a preferred application of the invention, the artificial tissue is used to provide a cardiac assist device (for either completely or partially assisting of the contraction function), either on the atrial and/or the ventricular level on one or both sides.

However, it is implicit that other applications may include assist or replacement of skeletal muscles, as well as temporary or permanent compression devices of hollow and solid organs.

### BRIEF DESCRIPTION OF THE DRAWINGS

The technical features of the present invention, in accordance with the above-mentioned aims, are set out in the claims herein and the advantages more clearly illustrated in the detailed description which follows, with reference to the accompanying drawings, which illustrate a preferred embodiment without limiting the scope of application, and in which:

FIG. 1 shows a schematic three dimensional structure of a contractile tissue according to the invention;

FIG. 2 shows a schematic representation of a contractile assist device applied to the atrial region of a heart.

### DESCRIPTION OF THE PREFERRED EMBODIMENTS

With reference to the accompanying drawings, a contractile tissue comprise a plurality of contractile elements 2 reciprocally connected by connecting elements 3, to form a three dimensional web 1.

Preferably, the contractile elements 2 may consist of Shape Memory Alloys actuators, which are able to contract when they are heated subject to an electric voltage, and then to recover an elongated state when cooled.

Connecting elements 3 may preferably consist of elastomeric material (i.e. polyurethane), but even rigid elements can be used to link the contractile elements 2 together.

Contractile elements 2 are linked in serial and/or parallel configuration in order to increase the displacement lenght of the web and/or the power of the contraction imposed in any predetermined direction X resulting form the spatial organization of elements 2. A single or a group of contractile elements 2 oriented in a generic direction X define a contractile unit 6. Depending on the structure of the contractile elements 2, it is also possible to link directly the contractile elements each other, avoiding the necessity of a connecting structure.

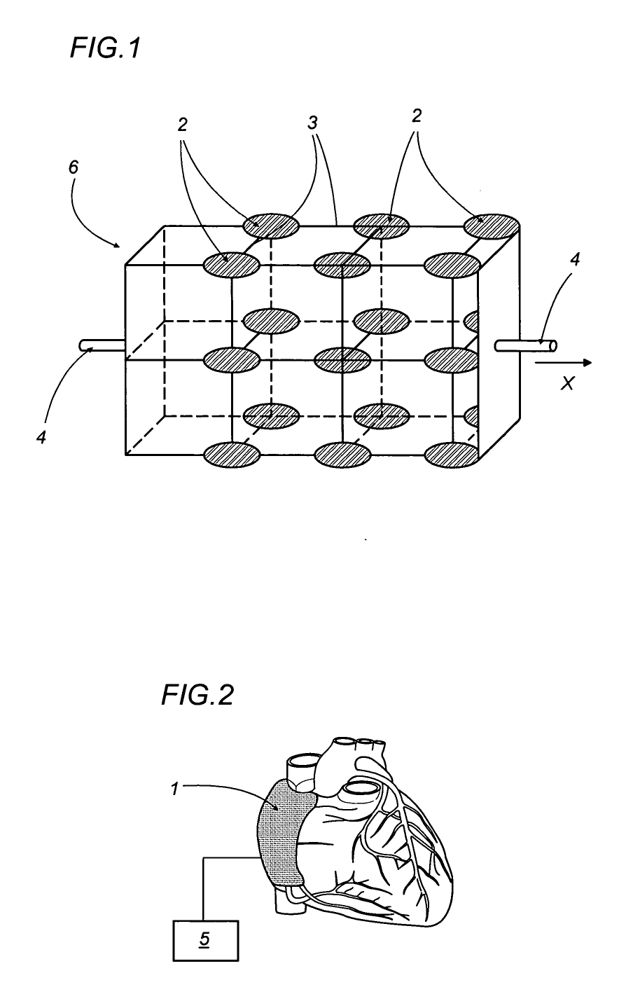

By example, possible actuators available on the market suitable to be used in the invention are the bio-metal actuators of BMX series (namely, Biometal actuators BMX20020) commercialized by Toki Corporation Inc., which are able to reversibly contract upon a controlled heating caused by the supply of an electric voltage. In the schematic view of FIG. 1, a unit 6 is provided of electrical terminal 4 which can be connected to an electric signals source to obtain a reversible contraction in direction X having a given frequence.

By example, the electric signal source can comprise a microprocessor control unit 5 driving one or more directional web 1 or one or more unit 6 of the same web in one or more directions.

A commercially available control unit are the driver boards and digital interfaces commercialized by NanoMuscles Inc., which are able to drive a plurality of actuators using standard 1.5 V batteries as power source.

Advantageously, the control unit 5 may be of a pacemaker size and therefore fully implantable.

According to an embodiment of the invention, unit 5 receive a predetermined sequence of pulses from a pulse generator and provides in response electric signals wich are supplied to the electric terminals of the individual contractile elements, or units, to control current direction, intensity and frequence of activation of contractile web.

The contraction can be synchronous, asynchronous, sequential or other, in order to achieve the most appropriate three dimensional deformation.

Under a further aspect of the invention, a cardiac contraction assist method comprises the steps of providing a three dimensional web consisting of electrically controlled contractile element spatially arranged according to at least one direction of contraction, fix the web to a cardiac region to be assisted in contraction (i.e. by suturing the web to the cardiac region external wall), and apply through a control unit an electric signal to electric terminals of the web to cause its controlled contraction in at least one direction.

Preferably, the cardiac region is an atrial region, and the web is partially or completely wrapped around the atrial region.

The method may further comprise a step of generation of a predetermined sequence of pulses to be transmitted to the control unit.

Preferably, all or some parameters among intensity, frequency and timing of the sequence of pulses are obtained on the basis of the heart beating detected by suitable sensors, consisting, in a possible example of a motion sensor placed on the heart surface.

In this case, the generated pulses are transmitted to the control unit for processing and then the unit—supplies the appropriate electric signals to the contractile web.

It is evident that the above device and method involve a number of advantages over the prior art assist device comprising electric or magnetic pumps, as well as over the known assist techniques to the compression functionality of the heart.

In particular:
use of electric or magnetic pumps is not longer necessary to assist blood circulation, and a smaller energy supply can be used, so that the device is totally implantable in the human body and can be driven by a pace maker size battery;
the natural biological lining of blood exposed surfaces is maintained, so that patients are set free from anticoagulation therapy;
the web can be manufactured, cut into any size or shape and then sutured or glued or anyhow attached to the cardiac region that needs assistance;
when using a motion sensor instead of a sensor of the electrical activity of the heart, the atrial assisted contraction can modulate ventricular filling as function of ventricular tele-diastolic volume improving the hemodynamic performance of the heart;
deformation cycles of Shape Memory Alloys or electroactive polymers elements can be repeated millions of times in a reliable way and they can work under tension, compression, bending or torsion.

The invention described may be used for evident industrial applications and can be subject to numerous modifications and variations without thereby departing from the scope of the inventive concept. Moreover, all the details of the invention may be substituted by technically equivalent elements.

1) an artificial contractile tissue for cardiac assist devices comprising:
a spatial arrangement of elements defining a three-dimensions contractile web, the contractile elements being able to perform a reversible contraction according to at least one direction, in order to determine a controlled contraction of the web in said at least one direction;
means to fixely apply the contractile web to a cardiac region of a patient.

2) The tissue according to claim 1, wherein said contractile elements are micro-actuators electrically activated.

3) The tissue according to claim 2, wherein said contractile elements comprise electrical terminals able to receive input electric pulses for contraction control in at least one predetermined direction.

4) The tissue according to claim 3, wherein said contractile elements are shape memory alloys elements.

5) The tissue according to claim 3, wherein said contractile elements are electro-active polymer elements.

6) The tissue according to claim 1, wherein said contractile web in embedded in an elastomeric matrix.

7) The tissue according to claim 1, wherein said means to apply the tissue to the cardiac region comprise a suturing of the web.

8) A cardiac assist contraction device comprising:
a contractile tissue consisting of a spatial organization of contractile elements defining a three-dimensions contractile web, the elements having electric terminals able to receive electric signals;
means to implant the contractile tissue to a cardiac region of a patient;
a control unit to supply electric signals driving the elements contraction according to at least one direction, in order to determine a controlled contraction of the web in said at least one direction.

9) The device according to claim 8, wherein said control unit is a low-voltage battery powered unit fully implantable in the human body.

10) The device according to claim 8, wherein said cardiac region is the atrial region.

11) The device according to claim 8, wherein said cardiac region is the ventricular region.

12) The device according to claim 8, wherein said contractile web is embedded in an elastomeric matrix.

13) The device according to claim 8, comprising a pulse generator supplying to said control unit a predetermined sequence of pulses.

14) The device according to claim 13, wherein said pulse generator comprise a heart beating sensor.

15) The device according to claim 14, wherein said hear beating sensor is a motion sensor.

16) A cardiac contraction assist method, comprising the steps of:
providing a three dimensional web consisting of electrically controlled contractile element spatially arranged according to at least one direction of contraction;
fix the web to a cardiac region to be assisted in contraction;
providing a control unit of the web;
apply through the control unit an electric signal to electric terminals of the web to cause its controlled contraction in at least one direction.

17) A method according to claim 16, wherein said cardiac region is an atrial region.

18) A method according to claim 16, wherein said cardiac region is the ventricular region.

19) A method according to claim 16, wherein said web is sutured to the cardiac region tissue.

20) A method according to claim 16, wherein said web is glued to the cardiac region tissue.

21) A method according to claim 16, further comprising a step of generation of a predetermined sequence of pulses to be transmitted to the control unit.

22) A method according to claim 16 restoring or improving the contractile performance of any biologic muscle.
